(19) United States
(12) Patent Application Publication
(10) Pub. No.: US 2026/0058112 A1
(43) Pub. Date: Feb. 26, 2026

(54) DEVICES AND SYSTEMS FOR USING, MONITORING AND CONTROLLING FAR ULTRAVIOLET-C RADIATION AND METHODS OF USING SUCH DEVICES AND SYSTEMS

(71) Applicant: Eden Park Illumination, Inc., Champaign, IL (US)

(72) Inventors: Sung-Jin Park, Champaign, IL (US); Zhihu Liang, Mahomet, IL (US); Jin Hoon Cho, Seongnam-Si (KR); Eui Ho Jung, Somerset, NJ (US); Caden J. Eagler, Urbana, IL (US)

(21) Appl. No.: 19/305,150

(22) Filed: Aug. 20, 2025

Related U.S. Application Data
(60) Provisional application No. 63/685,128, filed on Aug. 20, 2024.

(51) Int. Cl. H01J 61/95; A61L 2/10; H01J 61/32; H01J 61/56

(52) U.S. Cl. CPC H01J 61/95; A61L 2/10; H01J 61/327; H01J 61/56

(57) ABSTRACT

The present disclosure provides a device that may include a calibrated deep UV sensor head, control microelectronics, rechargeable batteries, displays, and/or network tools for data communication through the cloud. The disclosed device can detect and measure Far UV-C radiation, allowing information to be shared with users in remote locations. It may be compact, portable, and can be integrated into any Far UV-C devices or systems. Specifically designed for monitoring and potentially controlling Far UV-C radiation with wavelengths below 240 nm, this device may be ideal for use in various indoor settings where preventing overexposure to Far UV-C is crucial. Additionally, the measured intensities can be transmitted via common wireless communication protocols such as Wi-Fi, Bluetooth, GSM, and telecommunication networks.

### PRIORITY CLAIM

The present application claims priority to U.S. Provisional Application No. 63/685,128 filed Aug. 20, 2024, the disclosure of which is incorporated by reference herein in its entirety.

### GOVERNMENT RIGHTS IN INVENTION

This invention was made by an agency of the United States Government under a contract with an agency of the United States Government, US DOE contract (Award) #DE-SC0021718 (title: Development of Miniature Far UV-C Microplasma Lamps with Improved Safety Factors for Personal Protective Respirators). The government has certain rights in the invention.

### BACKGROUND

The present disclosure generally relates to devices and systems of monitoring and controlling ultraviolet (UV) radiation. The present disclosure also relates to methods of using such devices and systems.

During the pandemic, various technologies, including UV disinfection, were widely deployed to control infections and minimize airborne transmission of the coronavirus in public spaces. However, questions regarding the effectiveness of these techniques led international and U.S. federal entities (such as WHO, CDC, and FDA) to issue guidelines on their proper use. For UV disinfection, these guidelines emphasize both germicidal effectiveness and human safety. It is essential to confirm not only the performance of UV radiation (e.g., dosage and exposure time) for effective disinfection but also to ensure precise monitoring of exposure levels to prevent exceeding safety limits in occupied spaces.

Ultraviolet-C (UV-C) radiation (100-290 nm) has been used for disinfecting air and surfaces. UV-C disinfection systems have demonstrated their effectiveness in reducing the transmission of diseases, including viral infections like measles, mumps, SARS-COV2, and influenza, as well as bacterial infections such as tuberculosis, listeria, and Clostridioides difficile (C-diff). Conventional UV-C applications, often referred to as ultraviolet germicidal irradiation (UVGI), primarily utilize low-pressure mercury lamps (254 nm) or UV-C LED lamps (mainly in the 260-290 nm range).

The primary inactivation mechanism of UV-C radiation involves damaging the chemical bonds in nucleic acids (DNA and RNA), exploiting their maximum absorption band around 260 nm. However, these UV photons can also harm mammalian skin and eyes upon exposure, necessitating strict regulations for conventional UV-C systems to minimize human tissue exposure during air and surface disinfection.

Conventional UV-C disinfection technology is typically used in controlled environments where minimal or no human exposure is expected, as per governmental guidelines. Examples include UV ventilation systems, air purifiers, and UV sterilization units. In contrast, Far UV-C applications require extensive monitoring due to their potential for continuous prevention of airborne transmission and surface disinfection in occupied spaces.

Recent research has highlighted the unique properties of Far UV-C radiation (200-235 nm), particularly concerning human and animal safety. Due to the short wavelength-optical absorption relationship, Far UV-C photons do not penetrate or have significantly minimized penetration of the outermost layers of the skin (stratum corneum) and eyes (corneal epithelium). As a result, studies have shown no formation of photo-carcinogenic factors even with high exposure levels.

Furthermore, Far UV-C radiation has been found to be at least as effective as conventional UV-C radiation in inactivating a broad range of microbial and viral pathogens, including SARS-CoV-2 and other airborne-transmitted diseases. Given its combined safety and effectiveness, Far UV-C radiation is being considered for new disinfection applications that were not feasible with conventional UV technology. Specifically, it may allow for greater exposure of human tissues to UV output while maintaining safety, potentially revolutionizing the application of UV-C radiation for air and surface disinfection.

The FDA recommends that manufacturers of UV devices evaluate controls for time, UV radiation dose, and intensity, and validate cleaning and disinfection procedures. However, UV disinfection applications extend beyond individual devices to include tunnels, rooms, elevators, and other open-air or confined spaces where UV light is used to eliminate pathogens. Ideally, manufacturers should provide end-users with tools to assess the effectiveness of disinfection devices or UV sources in real-time, necessitating UV-C irradiance sensors with appropriate form factors, functionality, and the ability to provide real-time irradiance and dose measurements with high confidence. In health-sensitive environments such as medical or public health facilities, it is crucial to monitor the performance of Far UV-C light sources.

However, many existing devices and environments do not consistently monitor whether exposure and dose levels meet the necessary thresholds for pathogen deactivation. Evidence suggests that many commercially available UV-C irradiance sensors exhibit poor and varied performance, leading to high uncertainties in the effectiveness of ultraviolet germicidal irradiation (UVGI) devices and a limited understanding of irradiance levels reaching target areas for surface disinfection.

Far UV-C technology is increasingly being used for disinfection in occupied indoor spaces due to its enhanced safety profile. However, global safety regulations and guidelines require precise dosage control of these lights, especially in the presence of people.

While several Far UV-C detectors are commercially available, for example, from manufacturers like International Light Technology, Hamamatsu, and Gigahertz Optics, they are typically used for calibrated, high-accuracy intensity measurements (commonly referred to as “meters” rather than “sensors”) primarily for scientific or industrial purposes. These detectors are generally bulky, relatively expensive, and challenging to miniaturize for integration as a built-in feature within a product.

Chemical-based, passive Far UV-C color-changing indicator sheets are available. While they are low-cost and easy to use, they have significant drawbacks. For example, the color change occurs slowly, the detection sensitivity is very poor, and the sheets can be easily contaminated or affected by environmental factors.

### SUMMARY

The present disclosure addresses the problems and challenges of the conventional UV disinfection technology. The present disclosure generally relates to devices and systems of monitoring and controlling UV radiation. More specifically, the present disclosure relates to devices and systems of monitoring and controlling cloud based, miniature far UV-C radiation. Even more specifically, the present disclosure relates to miniature, portable or built-in, battery-powered, high sensitivity far UV-C sensor devices and systems with the Internet of Things (IOT) connectivity. The present disclosure also relates to methods of using such devices and systems.

An aspect of the present disclosure is a device comprising a housing and further comprising components, each of the components is positioned within the housing and/or mechanically connected to the housing, the components comprising:
a lamp that is a source of Far UV-C light;
a detector configured to continuously or periodically measure intensities of the Far UV-C light from the lamp; and
control microelectronics configured to perform automatic adjustments of the intensities of the Far UV-C light emitted by the lamp, based on the intensities of the Far UV-C light that are measured by the detector.

Another aspect of the present disclosure is a device for continuously or periodically measuring intensity of Far UV-C light emitted from a lamp, the device comprising:
a sensor module comprising a housing and further comprising components, the components comprising:
a unique sensor module identification code;
a detector configured to continuously or periodically measure intensities of the Far UV-C light;
control microelectronics configured to process the measured intensities of the Far UV-C light; and
a network module configured to communicate the measured intensities to storage, the storage accessible by one or more user devices for monitoring of the measured intensities of the Far UV-C.

Yet another aspect of the present disclosure is a method of inactivating a pathogen in air and/or on a surface, the method comprising:
using the device disclosed herein to emit the Far UV-C light from the lamp into and/or onto the air or the surface, to thereby inactivate the pathogen; and
performing automatic adjustments of the intensities of the Far UV-C light emitted by the lamp, based on the intensities of the Far UV-C light that are measured by the detector.

A further aspect of the present disclosure is a method of making the device disclosed herein, the method comprising at least one step selected from the group consisting of (i) connecting at least one of the lamp or the detector to the housing and (ii) mechanically connecting at least one of the lamp or the detector to the housing.

A yet further aspect of the present disclosure is a system for continuously or periodically measuring intensity of Far UV-C light, the system comprising:
one or more sensor modules, each of the sensor modules comprising: a unique sensor module identification code;
a detector configured to continuously or periodically measure intensities of the Far UV-C light;
control microelectronics configured to process the measured intensities of the Far UV-C light; and
a network module configured to communicate the measured intensities to storage, the storage accessible by one or more user devices for monitoring of the measured intensities of the Far UV-C.

Additional features and advantages are described in, and will be apparent from, the following Detailed Description and the Figures.

### BRIEF DESCRIPTION OF DRAWINGS

FIG. 1 shows a non-limiting example of the detection sensitivity of the semiconductor sensor chip used in the devices and systems according to the present disclosure in relation to the sensor's detection range.

FIG. 2 shows non-limiting examples of basic components of a Far UV-C monitoring and controlling device according to the present disclosure.

FIG. 3 shows a non-limiting example of an external, miniature Far UV-C calibrated sensor with communication capabilities according to the present disclosure. The digital display shows the irradiation level of the Far UV-C light detected and device status information.

FIG. 4 shows a non-limiting example of a smart lamp system with a built-in Far UV-C sensor integrated into the Far UV-C lamp fixture according to the present disclosure. The upper right square panel shown in the photograph is the microplasma Far UV-C lamp. This system also features a digital display that shows the intensity values and other optional functional statuses.

FIG. 5 demonstrates the concept of multipoint calibration for built-in Far UV-C lamps using an internally integrated Far UV-C sensor. This non-limiting example illustrates the calibrated irradiation intensities of Far UV-C at various distances from the lamp.

FIG. 6 is a conceptual demonstration of a multi-Far UV-C sensor array measurement for analyzing Far UV-C light distribution.

FIG. 7 shows a multi-Far UV-C sensor array housed in a spherical fixture for real-time measurement of omnidirectional, 3 dimensional integrated fluence and simultaneous, independent directional irradiance. The figure shows a CAD model of the spherical sensor and a photograph of the fabricated device (overall dimensions: 9.5 cm height×8.5 cm width×4.0 cm lateral plane). Six independent Far-UVC sensors are embedded, each oriented in a different direction. The figure also illustrates use of the system to track dynamic Far-UVC intensity within a conveyor setup equipped with multiple Far UV-C lamps for the application of disinfection or treatment to a mass volume in-line product processing.

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

All percentages are by weight of the total weight of the composition unless expressed otherwise. Similarly, all ratios are by weight unless expressed otherwise. As used herein, “about,” “approximately” and “substantially” are understood to refer to numbers in a range of numerals, for example the range of −10% to +10% of the referenced number, preferably-5% to +5% of the referenced number, more preferably-1% to +1% of the referenced number, most preferably-0.1% to +0.1% of the referenced number.

Furthermore, all numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used herein and in the appended claims, the singular form of a word includes the plural, unless the context clearly dictates otherwise. Thus, the references “a,” “an” and “the” are generally inclusive of the plurals of the respective terms. For example, reference to “an ingredient” or “a method” includes a plurality of such “ingredients” or “methods.” The term “and/or” used in the context of “X and/or Y” should be interpreted as “X,” or “Y,” or “X and Y.” Similarly, “at least one of X or Y” should be interpreted as “X,” or “Y,” or “both X and Y.”

Similarly, the words “comprise,” “comprises,” and “comprising” are to be interpreted inclusively rather than exclusively. Likewise, the terms “include,” “including” and “or” should all be construed to be inclusive, unless such a construction is clearly prohibited from the context. However, the embodiments provided by the present disclosure may lack any element that is not specifically disclosed herein. Thus, a disclosure of an embodiment defined using the term “comprising” is also a disclosure of embodiments “consisting essentially of” and “consisting of” the disclosed components.

Where used herein, the term “example,” particularly when followed by a listing of terms, is merely exemplary and illustrative, and should not be deemed to be exclusive or comprehensive. Any embodiment disclosed herein can be combined with any other embodiment disclosed herein unless explicitly indicated otherwise.

As used herein, “Far UV-C” light has a wavelength of 200-235 nm, for example about 222 nm.

### Embodiments

The present disclosure introduces a novel methodology for monitoring and controlling lamps that emit deep UV wavelengths in the electromagnetic spectrum, particularly in the Deep UV-C range, with a focus on Far UV-C wavelengths (200-235 nm, such as about 222 nm). Unlike conventional UV-C wavelengths that can penetrate the skin and damage DNA, particularly in mammalian tissue, these wavelengths are highly effective at inactivating pathogens in the air and on surfaces, without causing harm to human or animal skin or eyes upon exposure.

One aspect of the present disclosure is a device that may include a calibrated deep UV sensor head, control microelectronics, rechargeable batteries, displays, and/or network tools for data communication through the cloud. This device may be a miniature device. This device can detect and measure Far UV-C radiation. This device may also allow information to be shared with users in remote locations. A particularly preferred embodiment is compact, portable, and can be integrated into any Far UV-C device or system. Specifically designed for monitoring and potentially controlling Far UV-C radiation with wavelengths below 240 nm, this device may be ideal for use in various indoor settings where preventing overexposure to Far UV-C is crucial. Additionally, the measured intensities can be transmitted via common wireless communication protocols, such as Wi-Fi, Bluetooth, GSM, and telecommunication networks.

Embodiments of the device disclosed herein may include a miniature, portable or wall-mounted, battery-powered, high-sensitivity Far UV-C sensor with IoT connectivity. The device disclosed herein allows for real-time monitoring of Far UV-C radiation levels in indoor environments, such as hospitals, schools, and offices, and provides alerts when radiation levels exceed safe thresholds.

The device disclosed herein is designed for portability and easy installation, making it versatile and suitable for a wide range of settings.

The device disclosed herein can detect and measure the intensity of Far UV-C wavelengths, such as in the range of 200-235 nm, at low intensities, ranging from a few hundred μW/cm² to tens of nW/cm². The device disclosed herein can be used for monitoring Far UV-C radiation in various geometries and distances within indoor spaces.

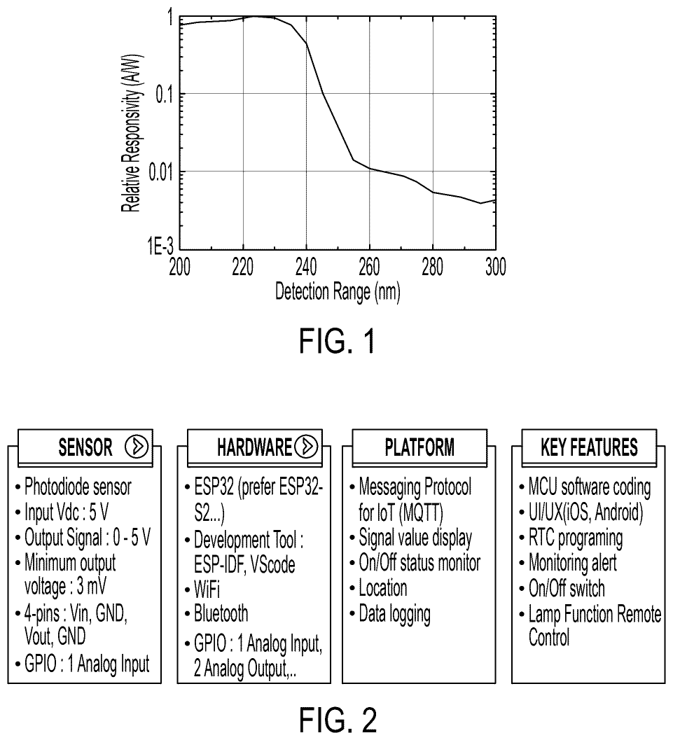

The detector chip used in the device disclosed herein can be sourced from a detector chip manufacturer. Any Far UV-C-sensitive semiconductor chips made from different chemical compositions can be used in the device disclosed herein, provided their performance meets the specified requirements for Far UV-C wavelengths. For example, an Aluminum Nitride (AlN) based detector chip can be used, and/or hexagonal-boron nitride (h-BN) or a doped diamond. This type of chip demonstrates high sensitivity to deep UV detection, as illustrated in FIG. 1. As shown in the figure, compared to conventional UV-C germicidal (UVGI) wavelengths longer than 250 nm, the detectivity of Far UV-C wavelengths significantly increases by more than two orders of magnitude at wavelengths below 240 nm.

The device disclosed herein can be built-in with the Far UV-C lamp or a stand-alone portable device, depending on user needs. When activated, the device may begin detecting Far UV-C radiation levels and transmit data via an IoT platform. The device disclosed herein can be configured to send alerts when radiation levels exceed a predetermined threshold. Additionally, the device disclosed herein can adjust its sensitivity to suit various indoor environments, ensuring accurate detection of Far UV-C radiation levels across different settings. The microprocessor of the device disclosed herein can also connect to an actuator or switching network (such as electronic relay) that can control the device or other physical devices related to the operation of Far UV-C radiating lamp or related devices.

In some embodiments, the device disclosed herein may be as a stand-alone, miniature monitoring sensor device that can be placed anywhere in the light irradiation field from a Far UV-C light source.

The accuracy of the device disclosed herein relies on its calibration against intensity standards. Given the limited availability of high-accuracy (NIST) standards for calibrating Far UV-C sensors, multiple standard references can be used to enhance reliability and accuracy.

Another aspect of the present disclosure is systems of Far UV-C sensors integrated with other sensors, such as those for moisture, ozone concentration, odor, VOC (volatile organic compounds) concentration, and potentially even biological contamination monitoring sensors (biosensors in airborne or surface). Such systems can provide significant and valuable data, enhancing the effectiveness of disinfection processes and improving overall performance and data accuracy.

A key advantage of the devices and systems provided herein is their wavelength selectivity specifically in the Far UV-C region (200-235 nm), with minimized sensitivity interference from other UV-C wavelengths. Conventional, commercially available UV-C meters (or sensors) typically use detector heads designed for maximum sensitivity at 254 nm or other wavelengths, often with a broad bandwidth sensitivity of at least 20 nm. To adapt these detectors, they often rely on bandpass filters, which can complicate the design and limit specificity.

In contrast, embodiments in the present disclosure offer one or more several distinct advantages:
1. Wavelength Selectivity: The device disclosed herein is finely tuned for the Far UV-C region, ensuring precise measurement without interference from other UV-C wavelengths.
2. Compact and Miniature Form Factor: The device disclosed herein is small and lightweight, making it easy to install in various Far UV-C application environments.
3. Simpler Operation: The device disclosed herein is easy to use, with a straightforward setup and operation process.
4. Battery-Powered and Portable: The device disclosed herein can be battery-operated, allowing it to be easily moved and deployed in different locations without the need for constant power sources.
5. IoT Connectivity: The device disclosed herein can features IoT connectivity, enabling remote monitoring and data analysis through existing network environments like Wi-Fi or Bluetooth. This allows for real-time monitoring and interfacing with other physical devices, facilitating comprehensive radiation safety management.
6. High Sensitivity and Accuracy: The device disclosed herein is capable of high-sensitivity detection, allowing users to measure Far UV-C radiation levels in real time with exceptional accuracy.
7. Low-cost: The device disclosed herein can be manufactured at low cost.
8. Functionality of multiple sensor arrays: The device can be configured with multiple sensor arrays in different orientations or arrangements to measure Far UV-C, allowing simultaneous readings from individual sensors.

These advantages make the device disclosed herein superior to existing radiation detectors, offering greater flexibility, precision, and ease of deployment in a wide range of applications.

Another aspect of the present disclosure is methods of using the device or system provided herein.

In some embodiments, the device disclosed herein can be used to measure indoor irradiation intensities (dosage) for the safety (human or animal exposure efficiency) or pathogen disinfection effectiveness. Using known data for the safety (human or animal exposure efficiency) and pathogen disinfection effectiveness from available scientific test results and/or agencies for human safety, the device disclosed herein can be easily calculated (internally) for the exposure status and required conditions to achieve both safety and effectiveness.

In some embodiments, the device disclosed herein can be used to monitor Far UV-C intensities at any indoor applications, especially health sensitive applications (such as hospitals, clinics, and nursing home etc.) and public spaces (such as airports, transportations and etc.).

In some embodiments, the device disclosed herein can be used for the monitoring and/or controlling of Far UV-C devices in operation.

Another aspect of the present disclosure is methods of monitoring and/or controlling Far UV-C radiation from lamps connected to the device disclosed herein.

In some embodiments, the device disclosed herein can be used to monitor and/or control flat and compact microplasma (or other form factor) Far UV-C lamps.

Yet another aspect of the present disclosure is methods of using and/or calibrating the device disclosed herein for microplasma Far-UV-C lamps.

In view of the disclosures herein, non-limiting embodiments include:

1. A device comprising a housing and further comprising components, each of the components is positioned within the housing and/or mechanically connected to the housing, the components comprising:
a lamp that is a source of Far UV-C light;
a detector configured to continuously or periodically measure intensities of the Far UV-C light from the lamp; and
control microelectronics configured to perform automatic adjustments of the intensities of the Far UV-C light emitted by the lamp, based on the intensities of the Far UV-C light that are measured by the detector.

2. A device for continuously or periodically measuring intensity of Far UV-C light emitted from a lamp, the device comprising:
a sensor module comprising a housing and further comprising components, the components comprising:
a unique sensor module identification code;
a detector configured to continuously or periodically measure intensities of the Far UV-C light;
control microelectronics configured to process the measured intensities of the Far UV-C light; and
a network module configured to communicate the measured intensities to storage, the storage accessible by one or more user devices for monitoring of the measured intensities of the Far UV-C.

3. The device of embodiment 1, wherein the control microelectronics are configured to perform the automatic adjustments to the lamp to thereby maintain the intensities of the Far UV-C light emitted from the lamp at a substantially consistent level.

4. The device of embodiment 2, wherein the one or more user devices are configured to perform automatic adjustments to the lamp based on the measured intensities of the Far UV-C light to thereby maintain the intensities of the Far UV-C light emitted from the lamp at a substantially consistent level.

5. The device of embodiment 1 or embodiment 2, wherein the detector is configured to detect the Far UV-C light at intensities ranging from 500 μW/cm² to 100 nW/cm².

6. The device of any of embodiments 1-5, wherein the control microelectronics comprise a microcontroller and further comprise an analog-to-digital converter (ADC), wherein the ADC is configured to convert an analog signal from the detector into a digital signal processed by the microcontroller, and preferably the microcontroller is configured for controlling the ADC.

7. The device of any of embodiments 1-6, further including a power supply, wherein the power supply comprises a rechargeable battery.

8. The device of any of embodiments 1-7, wherein the detector is part of a detector head that is part of the housing, and preferably the detector head is configured to mechanically rotate relative to at least one other part of the housing, for example to mechanically rotate at least 90 degrees relative to at least one other part of the housing.

9. The device of any of embodiments 1, 3 and 5-8, wherein the components further comprise a network module configured to conduct wireless communication identifying the intensities of the Far UV-C light that are continuously or periodically measured by the detector, preferably for cloud storage accessible from a location remote from the device.

10. The device of any of embodiments 1-9, wherein the components further comprise a display configured to provide visible indicia that indicate the intensities of the Far UV-C light measured by the detector.

11. The device of any of embodiments 1-10, wherein the lamp is a microplasma Far UV-C lamp.

12. The device of any of embodiments 1-11, wherein the control microelectronics comprise a microcontroller configured for at least one of (i) data processing, (ii) controlling at least one of the network module or the ADC, (iii) managing the power supply or (iv) monitoring aging and/or end-of-life status of the lamp.

13. The device of any of embodiments 1-12, wherein the detector is configured to monitor and measure the intensities of the Far UV-C light at multiple calibrated distances from the lamp, preferably comprising processing by the control microelectronics of multiple pre-measured reference standards of the lamp.

14. The device of embodiment 13, wherein the control microelectronics are configured to perform the automatic adjustments of the lamp to thereby maintain the intensities of the Far UV-C light emitted from the lamp at a substantially consistent level at each of the multiple calibrated distances, comprising a first substantially consistent level at a first calibrated distance and a second substantially consistent level at a second calibrated distance, wherein the first and second substantially consistent levels can be the same or different than each other.

15. The device of any of embodiments 1-14, wherein the components further comprise an additional detector, wherein the additional detector measures an intensity of light that is not in the Far UV-C wavelengths and preferably generates an alert when the intensity of light that is not Far UV-C meets a threshold.

16. The device of any of embodiments 1-15, wherein the lamp does not emit any light that is not Far UV-C.

17. The device of any of embodiments 1-16, wherein the detector comprises a photodiode comprising a Far UV-C-sensitive semiconductor chip.

18. The device of embodiment 17, wherein the Far UV-C-sensitive semiconductor chip comprises at least one of aluminum nitride (AlN), aluminum gallium nitride (AlGaN), hexagonal-boron nitride (h-BN) or a doped diamond.

19. The device of any of embodiments 1, 3 and 5-18, wherein the control microelectronics are configured to confirm the performance of the Far UV-C light from the lamp for effective disinfection.

20. The device of any of embodiments 1-19, wherein each of the components is positioned within the housing.

21 The device of any of embodiments 1-20, wherein the network module is configured to communicate an alert when the intensity of the Far UV-C light exceeds a threshold for occupied spaces.

22 The device of embodiment 2, wherein the housing of the sensor module is compact having dimensions no larger than 5 cm×5 cm×5 cm.

23. A method of inactivating a pathogen in air and/or on a surface, the method comprising:
using the device of any of embodiments 1-22 to emit the Far UV-C light from the lamp into and/or onto the air or the surface, to thereby inactivate the pathogen; and
performing the automatic adjustments of the intensities of the Far UV-C light emitted by the lamp, based on the intensities of the Far UV-C light that are measured by the detector.

24. The method of embodiment 23, wherein the device is portable or wall-mounted.

25. The method of embodiment 23 or embodiment 24, comprising human exposure to at least a portion of the Far UV-C light emitted by the lamp.

26. The method of any of embodiments 23-25, wherein the method is performed in an interior of a medical or public health facility or a laboratory.

27. The method of any of embodiments 23-27, comprising continuously monitoring whether the intensities of the Far UV-C light emitted by the lamp meet a threshold effective for pathogen deactivation.

28. A method of making the device of any of embodiments 1, 3 or 5-21, the method comprising at least one step selected from the group consisting of (i) connecting at least one of the lamp or the detector to the housing and (ii) mechanically connecting at least one of the lamp or the detector to the housing.

29. A system for continuously or periodically measuring intensity of Far UV-C light, the system comprising:
one or more sensor modules, each of the sensor modules comprising: a unique sensor module identification code;
a detector configured to continuously or periodically measure intensities of the Far UV-C light;
control microelectronics configured to process the measured intensities of the Far UV-C light; and
a network module configured to communicate the measured intensities to storage, the storage accessible by one or more user devices for monitoring of the measured intensities of the Far UV-C.

30. The system of embodiment 29, wherein the one or more user devices are configured to perform automatic adjustments to a Far UV-C lamp based on the measured intensities of the Far UV-C light to thereby maintain the intensities of the Far UV-C light emitted from the lamp at a substantially consistent level.

31. The system of embodiment 29 or embodiment 30, wherein the one or more user devices are configured to confirm the performance of the Far UV-C light from the lamp for effective disinfection.

32. The system of any of embodiments 29-31, wherein the one or more user devices are configured to communication an alert when the intensity of the Far UV-C light exceeds a threshold for occupied spaces.

33. The system of any of embodiments 29-32, further including a lamp, wherein the lamp is a source of Far UV-C light.

34. The system of any of embodiments 29-33, wherein the one or more sensor modules are configured to monitoring aging and/or end-of-life status of the lamp.

### EXAMPLES

The following non-limiting examples illustrate different aspects and/or embodiments of the devices and/or systems and/or methods of using such devices and/or systems according to the present disclosure.

### Example 1: Detection Sensitivity

FIG. 1 shows a non-limiting example of the detection sensitivity of the semiconductor sensor chip used in the devices and systems provided herein according to the present disclosure in relation to the detection range. FIG. 1 shows that the sensor is specialized for detecting Far UV-C wavelengths below 240 nm.

### Example 2: Basic Components of a Far-UV-C Monitoring and Control Device

Non-limiting examples of basic components and functionalities of a Far-UV-C monitoring and control device disclosed herein according to the present disclosure are listed in FIG. 2. The basic Far UV-C sensor device is a compact device with dimensions no larger than 5 cm(W)×5 cm(D)×5 cm(H).

It includes a Far UV-C detector, an analog-to-digital converter (ADC), a microcontroller, a battery, and an IoT device. The Far UV-C sensor device is a high-sensitivity solid-state device that detects Far UV-C radiation at a wavelength of 222 nm. The ADC converts the analog signal from the detector into a digital signal that can be processed by the microcontroller. The microcontroller handles data processing, controls the ADC and IoT module, and manages the power supply. The battery powers the sensor module for extended periods. The IoT device transmits data from the sensor device to a cloud-based server or local network, where it can be analyzed and displayed on a dashboard.

### Example 3: A miniature Far-UV-C Monitoring and Control Device

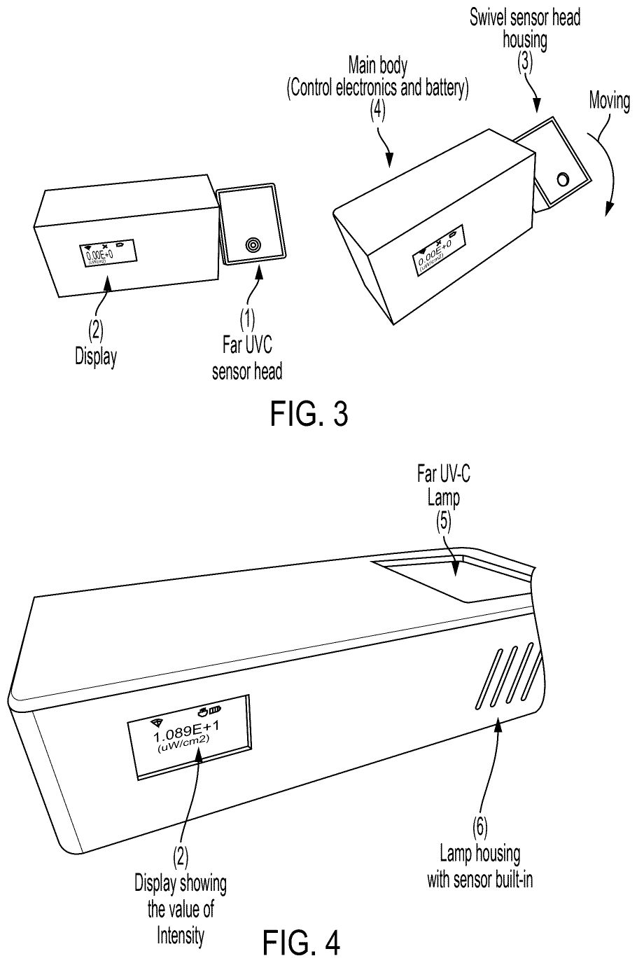

FIG. 3 shows a non-limiting example of a simple monitoring device with a miniature form factor according to the present disclosure. This device is powered by a rechargeable battery and suitable for mounting in any indoor space. For measurement convenience and enhanced accuracy, the detector head includes a swivel mechanism that allows for mechanical rotation, enabling reception angles of more than 90 degrees. Although this is an external device, its data can be transferred either wirelessly and/or through wired connections to the cloud, mobile devices (such as smartphones), and/or personal computers. Each detector is assigned a unique identification code, allowing multiple sensors to be monitored simultaneously by a single reading device. Additionally, since the data can be securely uploaded to cloud storage, authorized personnel can monitor the irradiation intensities of specific Far UV-C lamps from any location.

### Example 4: A Built-In Far UV-C Sensor Device Integrated in A UV-C Lamp

FIG. 4 shows a non-limiting example of a built-in Far UV-C sensor that is seamlessly integrated into a UV-C lamp, providing real-time monitoring of UV radiation levels directly at the source. This compact sensor is designed to fit within the lamp housing, allowing it to continuously detect and measure UV intensities without the need for external devices. The sensor operates by directly interfacing with the lamp's control system, enabling automatic adjustments based on the detected UV levels. This ensures that the lamp maintains optimal disinfection performance while safeguarding against overexposure.

Some key features of the built-in UV sensor include the following:
1) Compact Integration: The sensor is embedded within the lamp structure, making it unobtrusive and maintaining the lamp's design compact.
2) Real-Time Monitoring: The sensor continuously measures the UV radiation emitted by the lamp, providing instant feedback on intensity levels.
3) Automated Control: By integrating with the lamp's control system, the sensor can trigger (or modulate) adjustments to the lamp's output, ensuring consistent and safe Far UV-C exposure.
4) Data Communication: The sensor can transmit data to external systems, such as cloud storage or mobile devices, via wired or wireless communication protocols. This enables remote monitoring and control by authorized personnel.
5) Enhanced Safety: By constantly monitoring UV levels, the built-in sensor helps prevent overexposure, ensuring that the lamp operates within safe parameters, particularly in environments where human presence is common.
6) Calibration and Accuracy: The sensor is calibrated to detect accurate intensity of Far UV-C wavelengths, ensuring precise measurements that contribute to effective disinfection and safety compliance.
7) The sensor can monitor the aging or end-of-life status of the Far UV-C lamp, which is crucial for installations in health-sensitive applications. This information can be relayed to both the user and the vendor, allowing for timely replacement or maintenance to ensure continued safe and effective operation.

This built-in UV sensor transforms the lamp into a smart device, capable of self-regulating its output based on real-time data, making it ideal for use in environments where reliable and safe UV disinfection is critical.

### Example 5: Multi-calibration

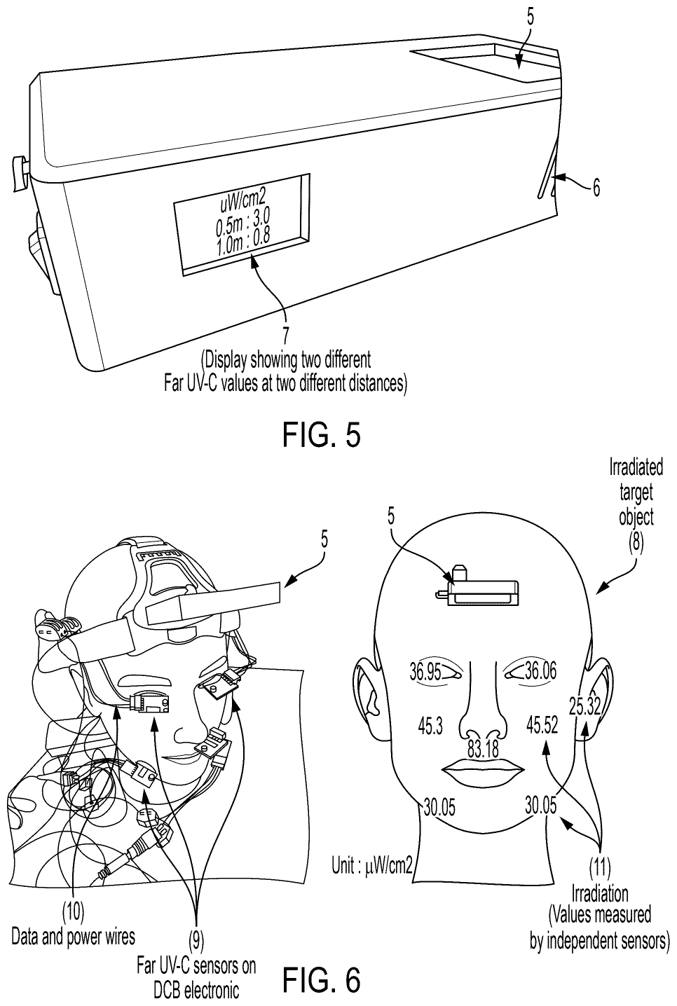

FIG. 5 shows a non-limiting example of a built-in UV sensor (inside the lamp fixture) with multi-calibration capabilities, allowing it to provide detailed information on UV irradiation across various distances.

This example illustrates the calibrated irradiation intensities of Far UV-C at various distances from the lamp. This advanced sensor is integrated directly into the lamp, enabling it to monitor and measure UV intensity not just at a single point, but across multiple calibrated distances from the lamp.

Some key features of this multi-calibrated built-in UV sensor include the following:
1) Multi-Distance Calibration: The Far UV-C sensor is calibrated to accurately measure UV radiation at different distances from the lamp (through a multiple pre-measured reference standards of the Far UV-C lamp), providing comprehensive data on irradiation intensity across a range of points. This is particularly useful in applications where precise control of UV exposure is necessary at varying distances.
2) Real-Time Monitoring and Feedback: The sensor continuously monitors the UV output, offering real-time data on how the intensity changes with distance. This information can be crucial for ensuring uniform disinfection in environments (such as humidity or air qualities) where UV coverage over a large area is required.
3) Smart Adjustments: The lamp can automatically adjust its output based on the data received from the sensor, ensuring that the desired UV intensity is maintained across all calibrated distances. This feature helps optimize the lamp's performance for consistent and effective disinfection.
4) Data Display and Communication: The system includes a digital display that shows real-time intensity readings at different distances, along with other operational status indicators (as shown in FIG. 6). The data can also be transmitted to external devices or cloud platforms for remote monitoring and analysis.
5) Enhanced Safety and Maintenance: By monitoring UV intensity across multiple distances, the sensor can detect irregularities or drops in performance that might indicate lamp aging or failure. Alerts can be sent to users and vendors, ensuring timely maintenance or replacement of the lamp to maintain safe and effective operation.
6) Application Versatility: This multi-calibrated sensor is ideal for complex environments where precise control of UV exposure is required, such as in healthcare facilities, laboratories, or other spaces where varying distances from the Far UV-C source are common.

### Example 6: A Multi-Far UV Sensor Measurement System

FIG. 6 shows a non-limiting example of a system employs multiple Far UV sensor arrays strategically positioned to analyze the light distribution across an irradiated target object. In FIG. 6, on the left, a mannequin is equipped with a sensor array mounted in various positions relative to the Far UV-C microplasma lamp positioned above the forehead. On the right, a computerized display shows the simultaneous sensor readings of Far UV-C irradiation at multiple points.

The data can be visualized as a 3D mapping, depending on the number of sensors in the array, providing a detailed analysis of the light distribution. These sensors work in unison temporally to provide a comprehensive mapping of Far UV-C intensity over the entire surface of the target object (herein, the mannequin simulating human head), ensuring uniform exposure and effective disinfection or treatment as well as securing skin safety from localized overexposure.

Some key features of this multi-Far UV sensor measurement system include the following:
1) Comprehensive Light Distribution Analysis: Multiple UV sensors are placed around the target object to measure the distribution of Far UV-C light across different areas. This setup allows for a detailed assessment of how evenly the UV radiation is being applied, identifying any areas of under- or over-exposure.
2) Real-Time Data Collection: The sensors continuously gather data on UV intensity at their respective positions, providing real-time feedback on the light distribution. This immediate information is crucial for making adjustments to ensure consistent and effective irradiation of the target object.
3) Uniformity Optimization: By analyzing the data from all sensors, the system can detect variations in light intensity across the target's surface. This information can be used to optimize the lamp's position, orientation, or output, ensuring that the entire object receives uniform UV exposure.
4) Computerized 3D Mapping Capability: The system can generate a 3D map of the UV light distribution across the target object, offering a visual representation of the irradiation pattern. This map helps in understanding how the UV light interacts with the object's shape and surface features, allowing for precise control and adjustments.
5) Automated Adjustments: Based on the sensor data, the system can automatically adjust the Far UV lamp's settings or the positioning of the target object to correct any discrepancies in light distribution, ensuring that all areas receive the appropriate level of UV exposure.
6) Application Versatility: This multi-sensor measurement system is ideal for applications where uniform UV exposure is critical, such as in sterilization processes, material testing, material reflectance analysis under Far UV radiation or Deep UV curing. It ensures that the target object is consistently and effectively treated, regardless of its size, shape, or surface complexity.

This advanced measurement system enhances the precision and effectiveness of Far UV-C irradiation by providing detailed insights into light distribution, enabling better control and ensuring optimal outcomes for the irradiated target object.

### Example 7: A multi-UV Sensor System for Both Safe and Harmful UV Wavelengths

Another non-limiting example is a multi-UV sensor system designed to measure the intensity of both safe and harmful UV wavelengths independently, providing a comprehensive analysis of overall safety and ensuring compliance with safety regulations. This system employs multiple sensors, each calibrated to detect specific UV wavelength ranges, allowing for accurate assessment of both beneficial and potentially harmful UV radiation. It can be integrated into Far UV-C lamp fixtures or used as a stand-alone system.

Some key features of the multi-UV sensor system include the following:
1) Dual-Wavelength Detection: The system is equipped with sensors that independently measure safe UV wavelengths (such as Far UV-C, which is effective for disinfection and safe for human exposure) and harmful UV wavelengths (such as conventional UV-C, which can cause skin and eye damage). This allows for precise monitoring of the full spectrum of UV radiation.
2) Safety Compliance Monitoring: By measuring the intensity of both safe and harmful UV wavelengths, the system provides real-time data that can be used to assess overall safety. This ensures that UV exposure levels remain within the limits set by safety regulations, helping to prevent overexposure and ensuring compliance with industry standards.
3) Real-Time Analysis and Alerts: The system continuously analyzes data from all sensors to calculate an overall safety factor. If harmful UV levels exceed safe thresholds, the system can trigger alerts to inform users or automatically adjust the UV source to reduce exposure.
4) Technical Artifact Detection: The system can detect and alert users to any technical anomalies, such as manufacturing errors or safety concerns related to Far UV-C radiation, including overintensity leakage of harmful UV-C radiation or angular radiation imperfections in lamp components. This ensures that any issues are addressed in real time.
5) Comprehensive Reporting: The system generates detailed reports on UV radiation levels, showing the distribution of safe and harmful wavelengths over time. These reports are valuable for safety audits, regulatory compliance, and optimizing the operation of UV-based systems.
6) Integration with Existing UV Systems: The multi-UV sensor system can be integrated with existing UV systems, providing a feedback loop that allows for dynamic adjustments based on real-time safety data. This integration ensures that UV systems operate safely while maintaining their effectiveness.
7) User-Friendly Interface: The system features an intuitive interface that displays real-time measurements, safety status, and compliance information. Users can easily monitor UV radiation levels and make informed decisions to maintain a safe environment. This multi-UV sensor system is an essential tool for any application where UV radiation is used, ensuring that the benefits of UV technology are harnessed without compromising safety.

### Example 8: a Multi-UV Sensor System for Real-Time Far-UVC Fluence Measurement in Multidimensional Irradiation Environments

Another non-limiting example is a multi-UV sensor system configured to measure total Far-UVC irradiation by integrating intensity values obtained in a three-dimensional (3D) field, while independently monitoring Far-UVC (safe band) of integrating overall Far-UVC. In industrial settings, Far-UVC is expected to be deployed in large-volume, in-line processes (e.g., food, agriculture, and pharmaceutical production). Achieving effective treatment typically requires highly uniform irradiation, which is often realized using multiple Far-UVC lamps in varied orientations and configurations. To increase throughput, targets are conveyed in motion; therefore, accurately evaluating the irradiation delivered to objects of specific sizes and shapes calls for a multi-UV sensor system with sensors oriented and calibrated to defined directions, replicating the actual treatment environment. To address this need, the present invention provides a new sensor form factor that enables efficient measurement.

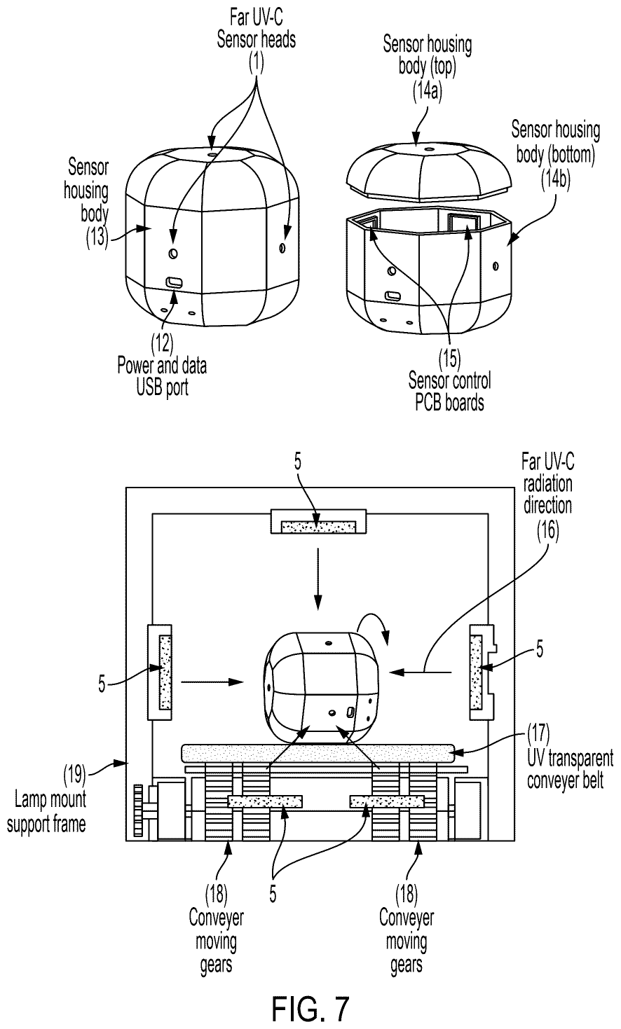

Key features of the multi-UV sensor system include:
1) Multi-directional detection: The standalone device integrates multiple sensors that independently measure Far-UVC and other UV bands at specified directions and angles, providing simultaneous per-orientation irradiance data and enabling calculation of total (3D-integrated) fluence on a target. FIG. 7 illustrates an example in which Far-UVC sensors are distributed uniformly around the device to capture irradiation from various angles concurrently.
2) Mobility via a unique form factor: The system employs a spherical (or 3 dimensional structure) housing that can be moved, or rolled, through a processing line, such as along conveyor belts (see FIG. 7). This geometry mimics the boundary conditions of representative targets intended for Far-UVC treatment, allowing the sensors to capture multi-directional irradiation during motion and to record the corresponding intensity values. Real-time analysis, remote monitoring, and wireless reporting: The system continuously aggregates data from all sensors to compute instantaneous irradiance and integrated fluence for the prospective target. The device is battery-powered and includes wireless communication and remote control of sensor operation. It can also report positional information within the treatment system and provide per-sensor readings as well as integrated values corresponding to overall optical fluence, thereby mapping the dose distribution along the conveyor path. These reports support evaluation of treatment efficacy for products undergoing disinfection.
3) Interfacing with UV treatment systems: The multi-UV sensor system integrates with existing irradiation equipment to provide a feedback loop for dynamic adjustment of lamp output and configuration, an important capability for maintaining treatment efficacy in high-throughput, in-line production.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

### FIGURE LEGEND

(1) Far UVC sensor head
(2) Display
(3) Swivel sensor head housing
(4) Main body (Control electronics and battery)
(5) Far UV-C Lamp
(6) Lamp housing with sensor built-in
(7) Display showing two different Far UV-C values at two different distances
(8) Irradiated target object
(9) Far UV-C sensors on DCB electronic
(10) Data and power wires
(11) Irradiation (Values measured by independent sensors)
(12) Power and data USB port
(13) Sensor housing body
(14a) Sensor housing body (top)
(14b) Sensor housing body (bottom)
(15) Sensor control PCB boards
(16) Far UV-C radiation direction
(17) UV transparent conveyer belt
(18) Conveyer moving gears
(19) Lamp mount support frame

1. A device comprising a housing and further comprising components, each of the components is positioned within the housing and/or mechanically connected to the housing, the components comprising:
a lamp that is a source of Far UV-C light;
a detector configured to continuously or periodically measure intensities of the Far UV-C light from the lamp; and
control microelectronics configured to perform automatic adjustments of the intensities of the Far UV-C light emitted by the lamp, based on the intensities of the Far UV-C light that are measured by the detector.

2. A device for continuously or periodically measuring intensity of Far UV-C light emitted from a lamp, the device comprising:
a sensor module comprising a housing and further comprising components, the components comprising:
a unique sensor module identification code;
a detector configured to continuously or periodically measure intensities of the Far UV-C light;
control microelectronics configured to process the measured intensities of the Far UV-C light; and
a network module configured to communicate the measured intensities to storage, the storage accessible by one or more user devices for monitoring of the measured intensities of the Far UV-C.

3. The device of claim 1, wherein the control microelectronics are configured to perform the automatic adjustments to the lamp to thereby maintain the intensities of the Far UV-C light emitted from the lamp at a substantially consistent level.

4. The device of claim 2, wherein the one or more user devices are configured to perform automatic adjustments to the lamp based on the measured intensities of the Far UV-C light to thereby maintain the intensities of the Far UV-C light emitted from the lamp at a substantially consistent level.

5. The device of claim 1, wherein the detector is configured to detect the Far UV-C light at intensities ranging from 500 μW/cm² to 100 nW/cm².

6. The device of claim 1, wherein the control microelectronics comprise a microcontroller and further comprise an analog-to-digital converter (ADC), wherein the ADC is configured to convert an analog signal from the detector into a digital signal processed by the microcontroller, and preferably the microcontroller is configured for controlling the ADC.

7. The device of claim 1, further including a power supply, wherein the power supply comprises a rechargeable battery.

8. The device of claim 1, wherein the detector is part of a detector head that is part of the housing, and preferably the detector head is configured to mechanically rotate relative to at least one other part of the housing, for example to mechanically rotate at least 90 degrees relative to at least one other part of the housing.

9. The device of claim 1, wherein the components further comprise a network module configured to conduct wireless communication identifying the intensities of the Far UV-C light that are continuously or periodically measured by the detector, preferably for cloud storage accessible from a location remote from the device.

10. The device of claim 1, wherein the components further comprise a display configured to provide visible indicia that indicate the intensities of the Far UV-C light measured by the detector.

11. The device of claim 1, wherein the lamp is a microplasma Far UV-C lamp.

12. The device of claim 1, wherein the control microelectronics comprise a microcontroller configured for at least one of (i) data processing, (ii) controlling at least one of the network module or the ADC, (iii) managing the power supply or (iv) monitoring aging and/or end-of-life status of the lamp.

13. The device of claim 1, wherein the detector is configured to monitor and measure the intensities of the Far UV-C light at multiple calibrated distances from the lamp, preferably comprising processing by the control microelectronics of multiple pre-measured reference standards of the lamp.

14. The device of claim 13, wherein the control microelectronics are configured to perform the automatic adjustments of the lamp to thereby maintain the intensities of the Far UV-C light emitted from the lamp at a substantially consistent level at each of the multiple calibrated distances, comprising a first substantially consistent level at a first calibrated distance and a second substantially consistent level at a second calibrated distance, wherein the first and second substantially consistent levels can be the same or different than each other.

15. The device of claim 1, wherein the components further comprise an additional detector, wherein the additional detector measures an intensity of light that is not in the Far UV-C wavelengths and preferably generates an alert when the intensity of light that is not Far UV-C meets a threshold.

16. The device of claim 1, wherein the lamp does not emit any light that is not Far UV-C.

17. The device of claim 1, wherein the detector comprises a photodiode comprising a Far UV-C-sensitive semiconductor chip.

18. The device of claim 17, wherein the Far UV-C-sensitive semiconductor chip comprises at least one of aluminum nitride (AlN), aluminum gallium nitride (AlGaN), hexagonal-boron nitride (h-BN) or a doped diamond.

19. The device of claim 1, wherein the control microelectronics are configured to confirm the performance of the Far UV-C light from the lamp for effective disinfection.

20. The device of claim 1, wherein each of the components is positioned within the housing.

21. The device of claim 1, wherein the network module is configured to communication an alert when the intensity of the Far UV-C light exceeds a threshold for occupied spaces.

22. The device of claim 2, wherein the housing of the sensor module is compact having dimensions no larger than 5 cm×5 cm×5 cm.

23. A method of inactivating a pathogen in air and/or on a surface, the method comprising:
using the device of claim 1 to emit the Far UV-C light from the lamp into and/or onto the air or the surface, to thereby inactivate the pathogen; and
performing the automatic adjustments of the intensities of the Far UV-C light emitted by the lamp, based on the intensities of the Far UV-C light that are measured by the detector.

24. The method of claim 23, wherein the device is portable or wall-mounted.

25. The method of claim 23, comprising human exposure to at least a portion of the Far UV-C light emitted by the lamp.

26. The method of claim 23, wherein the method is performed in an interior of a medical or public health facility or a laboratory.

27. The method of claim 23, comprising continuously monitoring whether the intensities of the Far UV-C light emitted by the lamp meet a threshold effective for pathogen deactivation.

28. A system for continuously or periodically measuring intensity of Far UV-C light, the system comprising:
one or more sensor modules, each of the sensor modules comprising:
a unique sensor module identification code;
a detector configured to continuously or periodically measure intensities of the Far UV-C light;
control microelectronics configured to process the measured intensities of the Far UV-C light; and
a network module configured to communicate the measured intensities to storage, the storage accessible by one or more user devices for monitoring of the measured intensities of the Far UV-C.

29. The system of claim 28, wherein the one or more user devices are configured to perform automatic adjustments to a Far UV-C lamp based on the measured intensities of the Far UV-C light to thereby maintain the intensities of the Far UV-C light emitted from the lamp at a substantially consistent level.

30. The system of claim 28, wherein the one or more user devices are configured to confirm the performance of the Far UV-C light from the lamp for effective disinfection.

31. The system of claim 28, wherein the one or more user devices are configured to communicate an alert when the intensity of the Far UV-C light exceeds a threshold for occupied spaces.

32. The system of claim 28, further including a lamp, wherein the lamp is a source of Far UV-C light.

33. The system of claim 28, wherein the one or more sensor modules are configured to monitoring aging and/or end-of-life status of the lamp.
